(19) United States
(12) Patent Application Publication
(10) Pub. No.: US 2026/0038683 A1
(43) Pub. Date: Feb. 5, 2026

(54) COMPUTING SYSTEM CONFIGURED TO FACILITATE MULTI-PARTY ADJUDICATION OF CARDIAC EPISODES

(71) Applicant: Medtronic, Inc., Minneapolis, MN (US)

(72) Inventors: Kelvin Mei, Minneapolis, MN (US); Tarek D. Haddad, Minneapolis, MN (US); Charles A. Sarbib-Brown, New Brighton, MN (US); Kyle R. Zablocki, Jamesburg, NJ (US)

(21) Appl. No.: 19/099,969

(22) PCT Filed: Aug. 15, 2023
(86) PCT No.: PCT/US2023/030242

§ 371 Date: Jan. 30, 2025

Related U.S. Application Data
(60) Provisional application No. 63/371,482, filed on Aug. 15, 2022.

(51) Int. Cl. G16H 40/67; G16H 10/60; G16H 50/20; G16H 80/00

(52) U.S. Cl. CPC G16H 40/67; G16H 10/60; G16H 50/20; G16H 80/00

(57) ABSTRACT

Techniques are described for facilitating multi-party adjudication of cardiac episodes. An example system includes memory storing instructions and processing circuitry configured to execute those instructions to receive episode data of a cardiac episode from a medical device. The processing circuitry is configured to determine a region of the episode data based on input from a primary adjudicator and to display a notification to a secondary adjudicator. The processing circuitry is further configured to display the region of episode data to the secondary adjudicator and to receive and transmit input from the secondary adjudicator.

This application claims the benefit of U.S. Provisional Application Ser. No. 63/371,482, filed Aug. 15, 2022, which is entitled, “COMPUTING SYSTEM CONFIGURED TO FACILITATE MULTI-PARTY ADJUDICATION OF CARDIAC EPISODES” and is hereby incorporated by reference in its entirety.

### FIELD

This disclosure generally relates to medical devices and, more particularly, to review and adjudication of episode data collected by medical devices.

### BACKGROUND

Medical devices may be used to monitor physiological signals of a patient. Such devices include implantable or wearable medical devices, as well as a variety of wearable health or fitness tracking devices. For example, some medical devices are configured to sense cardiac electrogram (EGM) signals, e.g., electrocardiogram (ECG) signals, indicative of the electrical activity of the heart via electrodes. Some medical devices are configured to detect occurrences of cardiac arrhythmia, often referred to as episodes, based on the cardiac EGM and, in some cases, data from additional sensors. Example arrhythmia types include asystole (or pause), bradycardia, ventricular tachycardia, supraventricular tachycardia, wide complex tachycardia, atrial fibrillation, atrial flutter, ventricular fibrillation, atrioventricular block, premature ventricular contractions, and premature atrial contractions. The medical devices may store the cardiac EGM and other data collected during a time period including an episode as episode data. The medical device may also store episode data for a time period in response to user input, e.g., input from the patient indicating concern that an arrhythmia episode may be occurring. Such acute health events involving cardiac arrhythmias may be associated with poor patient outcomes, e.g., death.

A computing system may obtain episode data from medical devices to allow a clinician or other user to review the episode. A clinician may diagnose a medical condition of the patient based on identified occurrences of cardiac arrhythmias within the episode. In some examples, one or more adjudicating clinicians or other adjudicators may review episode data to annotate the episodes, including determining whether arrhythmias detected by the medical device actually occurred, to prioritize the episodes and generate reports for further review by the caregiving clinician that prescribed the medical device for a patient or is otherwise responsible for the care of the particular patient.

### SUMMARY

In general, the disclosure describes techniques for streamlining the adjudication of cardiac episodes between adjudicators, e.g., a primary adjudicator and a secondary adjudicator. Cardiac episode data may be obtained from a medical device measuring and storing physiological signals, which a computing system may subsequently obtain. For example, a medical device configured to sense EGM signals may detect a potential arrhythmia episode of a patient. Adjudication of cardiac episodes may include a human adjudicator viewing the episode data, such as one or more segments of cardiac EGM data, e.g., ECG data, stored by the medical device in response to detecting the episode or in response to user input, on a display of a computing device. Based on the adjudicator's expertise in reviewing episode data and determining cardiac episodes based on such data, the adjudicator may determine whether one or more arrhythmias or other episodes of various types occurred-in some cases affirming, contradicting, or supplementing the episode detection by the medical device.

In some cases, cardiac episodes are adjudicated by multiple adjudicators. For example, primary adjudicators, such as nurses, nurse practitioners (NPs), or electrophysiologists (EPs) may perform an initial, and in the case of some episodes only, adjudication of the episode data. As an example, only one adjudication may be performed if a primary adjudicator is confident in the adjudication. If the primary adjudicator is unsure about a particular episode, a secondary adjudicator, who may be more experienced, such as a cardiologist, may be asked to review the episode and decide whether the episode detected by the medical device occurred.

However, current workflows for a primary adjudicator to send an ECG/EGM of interest to a secondary adjudicator are ineffective and highly manual. In some cases, current workflows involve printing out several pages of data and waiting outside the secondary adjudicator's door or interrupting a meeting to get the secondary adjudicator's attention and approval, sometimes multiple times a day. In other cases, the secondary adjudicator may examine an ECG/EGM of interest via unsecured messaging platforms, e.g., WhatsApp™, or the secondary adjudicator may need to access a patient's electronic health record (EHR) for examination. These interruptions and waiting periods are problematic for both primary and secondary adjudicators. The described highly manual and ineffective methods of sending an ECG/EGM of interest to a secondary adjudicator can force a primary adjudicator to neglect other important work-related duties. For example, in the event the primary adjudicator needs to wait for extended periods outside the secondary adjudicator's door to share the ECG/EGM data, the primary adjudicator may be unavailable to care for other patients.

Additionally, the current methods being used to share ECG/EGM data may involve printing or saving an entire data collection session, which is relatively long (e.g., several minutes of data) when compared to the quantity of data needed to make a diagnosis, e.g., 20 seconds of data. In the case of the printed report being used to send ECG/EGM data, the secondary adjudicator must flip through several pages of data to find areas of interest and make a diagnosis, and in the case of the data being uploaded to a patient's EHR, the secondary adjudicator may need to scroll through all the data of the episode recording rather than the important section(s). Due to the excess data being transferred to the secondary adjudicator, the efficiency of a secondary adjudicator's assessment may be lower than if only the essential or most helpful portions of the episode were shown.

The techniques of the disclosure described herein may provide specific improvements to the field of cardiac arrhythmia classification and adjudication. For example, a computing system configured according to this disclosure may include operating a server and one or more computing devices to improve efficiency of review and reduce the overall burden of episode review. In examples in which the techniques of this disclosure involve pinpointing specific portions of data to be reviewed, the chance of misdiagnosing a patient due to the burden of sifting through high volumes of data may be lowered. Additionally, adjudicators may be able to analyze more episode data and therefore provide better care to more patients. As such, a patient may be more likely to receive appropriate care in a timely manner, which may lead to better patient outcomes.

The medical device system may include a remote patient monitoring system. The remote patient monitoring system may inform a primary adjudicator of episode data and associated diagnoses, e.g., asystole, atrial fibrillation, tachycardia. Based on the primary adjudicator's experience and confidence in diagnosis, the primary adjudicator may determine the episode data should be examined by the secondary adjudicator. According to the techniques of this disclosure, the system may send the episode data to the secondary adjudicator using a remote patient monitoring system based on input from the primary adjudicator.

In one example, the primary adjudicator may send important portion(s) of the episode data and question(s) to the secondary adjudicator. The question(s) may be typed by the primary adjudicator, or the primary adjudicator may select one or more questions from a list of predefined questions to further streamline the adjudication process. The primary adjudicator may additionally be prompted to assign a priority level to the message sent to the secondary adjudicator indicating the level of urgency to inform the secondary adjudicator regarding which data to prioritize examining.

In another example, the portions of episode data most likely to contain the patient state being examined, e.g., atrial fibrillation or any other arrhythmia, may be selected using artificial intelligence (AI) or machine learning (ML) models. Additionally, the question options provided to the primary adjudicator may be selected from a longer list using AI or ML models to streamline question selection.

In another example, a model may be used to provide a confidence level regarding the medical device's detection of the cardiac event. For example, an output of confidence in the form of a percent may be provided, e.g., 85% confident that an atrial fibrillation episode occurred.

In some examples, the secondary adjudicator may receive a notification on his/her computing device, e.g., smartphone, and may then access the episode data via a secure application or secure web browser. The secondary adjudicator may then determine whether a concerning cardiac event e.g., tachyarrhythmia, occurred and type or select from a dropdown a reply to the primary adjudicator's question(s). The secondary adjudicator's opinion may then be sent to a database and saved. By streamlining the process of communication between primary and secondary adjudicators, the techniques of this disclosure may facilitate efficient verification of suspected health episodes. As such, patient outcomes may be improved.

In an example, a computing system comprising memory storing instructions and processing circuitry of one or more devices, including a server, a computing device of a primary adjudicator, and a computing device of a secondary adjudicator, is configured to execute those instructions to: receive, at the server, episode data of a cardiac episode from a medical device; identify, by the server, a region of the episode data; provide, via the computing device of the secondary adjudicator, a notification to the secondary adjudicator; display, in response to the secondary adjudicator interacting with the computing device of the secondary adjudicator in response to the notification, the region of the episode data onto the display of the computing device of the secondary adjudicator; receive, by the computing device of the secondary adjudicator, an input; and transmit, by the computing device of the secondary adjudicator and to the server, the input received from the secondary adjudicator in response to the episode data.

In another example, a method of operating a computing system to facilitate adjudication of cardiac events between a primary adjudicator and a secondary adjudicator includes: receiving, at a server, episode data of a cardiac episode from a medical device; identifying, by the server, a region of the episode data; providing, via a computing device of the secondary adjudicator, a notification to the secondary adjudicator; displaying, in response to the secondary adjudicator interacting with the computing device of the secondary adjudicator in response to the notification, the region of the episode data onto the display of the computing device of the secondary adjudicator; receiving, by the computing device of the secondary adjudicator, an input; and transmitting, by the computing device of the secondary adjudicator and to the server, the input received from the secondary adjudicator in response to the episode data.

In another example, a non-transitory computer readable storage medium comprises program storing instructions that, when executed by processing circuitry of a computing system comprising a server, a computing device of a primary adjudicator, and a computing device of a secondary adjudicator, cause the processing circuitry to perform a method comprising: receiving, at the server, episode data of a cardiac episode from a medical device; identifying, by the server, a region of the episode data; providing, via a computing device of the secondary adjudicator, a notification to the secondary adjudicator; displaying, in response to the secondary adjudicator interacting with the computing device of the secondary adjudicator in response to the notification, the region of the episode data onto a display of the computing device of the secondary adjudicator; receiving, by the computing device of the secondary adjudicator, an input; and transmitting, by the computing device of the secondary adjudicator and to the server, the input received from the secondary adjudicator in response to the episode data.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured to transmit physiological monitoring data to a plurality of computing devices, in accordance with one or more of the techniques of this disclosure.

FIG. 2 is a flow diagram illustrating an example of the techniques for cardiac episode adjudication including operation of a software interface, which is accessible to primary adjudicators, and a phone application, which is accessible to clinicians, as well as some examples of information the software interface and phone application may require as inputs from both adjudicators.

FIG. 3 is a diagram illustrating an example of cardiac EGM data of a cardiac episode.

FIG. 4 illustrates an example user interface including a zoomed in region of episode data.

FIG. 5 is a conceptual diagram illustrating an example software interface page for primary adjudicators to enter information related to the episode data.

FIG. 6 is a conceptual diagram illustrating example interfaces for a secondary adjudicator on which the analysis by the secondary adjudicator can be performed.

FIG. 7A is a conceptual diagram illustrating an example interface for primary adjudicators to make initial adjudications based on episode data and to decide whether to send episode data to a secondary adjudicator.

FIG. 7B is a conceptual diagram illustrating a pop-up interface resulting from a selection in the interface of FIG. 7A.

FIG. 8 is a conceptual diagram illustrating an example interface of and process by which a secondary adjudicator receives a notification that a new adjudication of cardiac episode data is needed and securely accesses a computing device.

FIG. 9 is a conceptual diagram illustrating an example user interface for secondary adjudicators to analyze cardiac episode data and make an assessment.

Like reference characters refer to like elements throughout the figures and description.

### DETAILED DESCRIPTION

A variety of types of implantable and external medical devices detect cardiac episodes based on sensed cardiac EGMs or ECGs (hereinafter “cardiac EGMs”) and, in some cases, other physiological parameters. External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic, Inc., of Dublin, Ireland. Such external devices may facilitate relatively longer-term monitoring of patients during normal daily activities, and may periodically transmit collected data, e.g., episode data for detected arrhythmia episodes, to a remote patient monitoring system, such as the Medtronic CareLink™ Network.

Implantable medical devices (IMDs) also sense and monitor cardiac EGMs and detect arrhythmia episodes. Example IMDs that monitor cardiac EGMs include pacemakers and implantable cardioverter-defibrillators, which may be coupled to intravascular or extravascular leads, as well as pacemakers with housings configured for implantation within the heart, which may be leadless. Some IMDs that do not provide therapy, e.g., implantable patient monitors, sense cardiac EGMs. One example of such an IMD is the Reveal LINQ™ or LINQ II™ Insertable Cardiac Monitor (ICM), available from Medtronic, Inc., which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic CareLink™ Network. While the techniques of this disclosure are discussed primarily with respect to cardiac IMDs and cardiac health, these techniques may be applied to other devices, including other IMDs and/or wearable devices which may be used to monitor other physiological parameters (e.g., non-cardiac) indicative of the health of the patient.

By uploading episode data from medical devices and distributing the episode data to various users, such network services may support centralized or clinic-based arrhythmia episode review, annotation, and reporting. The episode data may include the reason the medical device recorded the episode data, e.g., whether the episode was “device triggered,” meaning the medical device recorded the episode data in response to detection of an arrhythmia, or “patient triggered,” meaning the medical device recorded the episode data in response to input from the patient or another user. The episode data may include an indication of one or more arrhythmias that the medical device detected during the episode, e.g., the detection of which triggered the medical device to record the episode data. The episode data may also include data collected by the medical device during a time period including time before and after the instant the medical device determined the one or more arrhythmias to have occurred. The episode data may include the digitized cardiac EGM during that time period, heart rates or other parameters derived from the EGM during that time period, and any other physiological parameter data collected by the medical device during the time period. In some cases, although the medical device collected the episode data in response to detecting one or more arrhythmias, the review process may determine the medical device falsely detected one or more of the arrhythmias in an episode.

An IMD may interact with other devices. The IMD may communicate with an external device, such as a smart phone, and the external device may communicate with a remote patient monitoring system. Additionally, or alternatively, the IMD may communicate with the remote patient monitoring system, and the remote patient monitoring system may communicate with computing devices or other external devices. One or more servers with storage and processing circuitry, e.g., a cloud computing system, may implement the remote patient monitoring system and be used to analyze the data being uploaded by the IMD. The data stored by the patient monitoring system may be accessed by a computing device and displayed to a primary adjudicator via a user interface. The primary adjudicator may then perform an initial adjudication of the cardiac event.

In some examples, the primary adjudicator may be confident in the adjudication, in which case, the adjudication process may end.

In other examples, the primary adjudicator may not be confident in the adjudication and may decide to request that a secondary adjudicator perform a second adjudication. In this event, the primary adjudicator may use the remote patient monitoring interface to select a portion of episode data to send to the secondary adjudicator. The primary adjudicator may also choose to ask question(s), which will be sent along with the episode data. In some examples, the primary adjudicator may be assisted in these selections by AI or ML models.

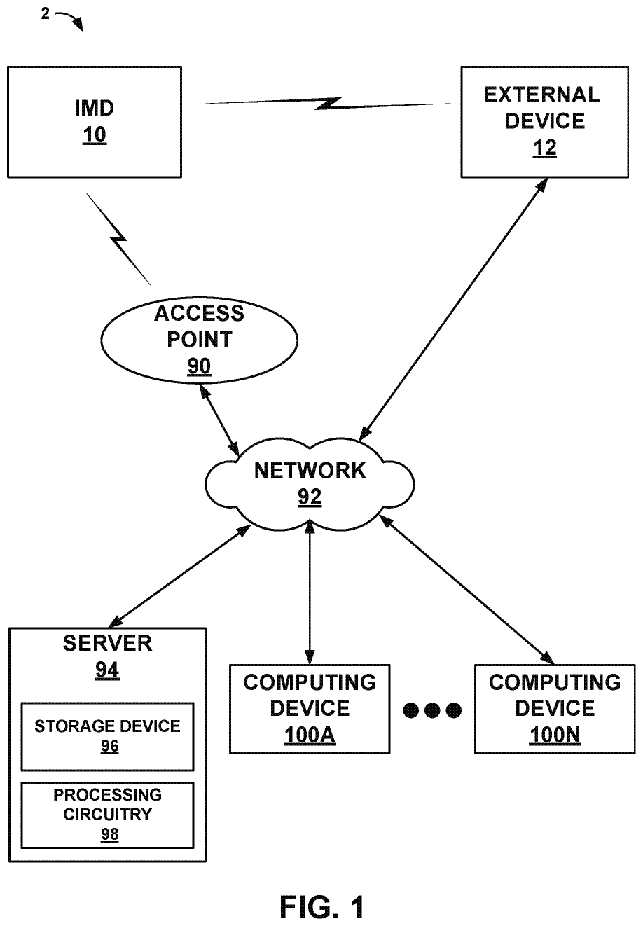

FIG. 1 is a block diagram illustrating an example computing system 2 configured to implement the techniques of this disclosure for facilitating multi-party adjudication of cardiac episodes. In the example illustrated by FIG. 1, system 2 may include an IMD 10 in communication with an external device 12 and an access point 90. External device 12 and access point 90 may further communicate with a network 92 which communicates with a server 94 and computing device(s) 100A-100N (collectively a “plurality of computing devices 100”).

IMD 10 may be implanted in a patient. IMD 10 may correspond to an ICM, or other IMDs, such as other implantable monitors, implantable pacemakers, defibrillators, neurostimulators, or substance delivery devices. In some examples, system 2 may include multiple IMDs 10 implanted in multiple patients. Additionally, or alternatively, to include IMD 10, system 2 may include external medical devices or any type of device configured to store episode data, e.g., in response to detecting a cardiac episode of a patient.

System 2 may include an external device 12, an access point 90, a network 92, a server 94, and one or more other computing devices 100A-100N, which may be interconnected and may communicate with each other through network 92. External device 12 is a computing device configured for wireless communication with IMD 10. External device 12 may be configured to communicate with server 94 via network 92. External device 12 may take the form of a tablet or smartphone. In some examples, external device 12 may provide a user interface and allow a user to interact with IMD 10. In some examples, external device 12 may function as a programmer for IMD 10. External device 12 may be a dedicated device for interacting with IMD 10 or may be a general purpose device configured with software for interaction with IMD 10. External device may be a smartphone or other computing device of the patient in which the IMD is implanted or may be a tablet or other computing device used by a clinician to interact with the IMD in a clinic.

Although not illustrated in FIG. 1, IMD 10 may include electrodes and other sensors to sense physiological signals and may collect and store sensed physiological data based on the signals and detect events based on the data. While this disclosure primarily discusses a sensing device as being an implantable medical device, IMD 10, in some cases the sensing device may be implemented as an external medical device or a wearable device rather than an IMD. As such, the techniques discussed herein may be practiced by an external medical device or a wearable device as well and the functionality attributed to IMD 10 may be practiced by an external medical device or a wearable device and still fall within the scope of this disclosure.

External device 12 may be used to retrieve data from IMD 10 and may transmit the data to server 94 via network 92. The retrieved data may include values of physiological parameters measured by IMD 10, indications of episodes of arrhythmia or other maladies detected by IMD 10, episode data collected for episodes, and other physiological signals recorded by IMD 10. The episode data may include EGM segments recorded by IMD 10, e.g., due to IMD 10 determining that an episode of arrhythmia or another malady occurred during the segment, or in response to a request to record the segment from the patient or another user. Access point 90 may be configured to similarly facilitate communication between IMD 10 and server 94 but need not include a user interface in some examples. Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient.

Server 94 may comprise computing devices configured to allow users to interact with IMD 10, or data collected from IMD 10, via network 92. In some cases, server 94 may be configured to provide a secure storage site for data that has been collected from IMD 10 and/or external device 12. In some cases, server 94 may be configured to provide a cloud-computing system. In some cases, server 94 may assemble data in web pages or other documents for viewing by trained professionals, such as episode adjudicators or other clinicians, via computing devices 100. One or more aspects of the illustrated system of FIG. 1 may be implemented with general network technology and functionality, which may be similar to that provided by the Medtronic CareLink™0 Network.

In some examples, one or more of computing devices 100 may be a personal computer, tablet, smartphone, or other computing device. A clinician may access data collected by IMD 10 through a computing device 100, such as when the patient is in in between clinician visits, to check on a status of a medical condition. Additionally, an adjudicator may access data collected by IMD 10 through a computing device, of computing devices 100, to adjudicate episodes, according to one or more techniques described herein. Computing devices 100, external device 12, and access point 90 may communicate with IMD 10 and each other according to Bluetooth®, Bluetooth® Low Energy (BLE), or other wireless communication protocols, as examples.

In the example illustrated by FIG. 1, server 94 includes a storage device 96, e.g., to store data retrieved from IMD 10, and processing circuitry 98. Although not illustrated in FIG. 1, any of computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, digital signal processors (DSPs), application-specific integrated circuits (ASICs), field-programmable gate arrays (FPGAs), or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuity of computing devices 100 may implement any of the techniques described herein for adjudication of cardiac episodes.

Storage device 96 may include a computer-readable storage medium or computer-readable storage device. In some examples, memory 96 includes one or more of a short-term memory or a long-term memory. Storage device 96 may include, for example, random-access memory (RAM), dynamic random-access memory (DRAM), static random-access memory (SRAM), magnetic discs, optical discs, flash memories, or forms of erasable programmable read-only memory (EPROM) or electrically erasable programmable read-only memory (EEPROM). In some examples, storage device 96 is used to store data indicative of instructions for execution by processing circuitry 98.

The techniques of this disclosure may provide a streamlined step-by-step system, in some cases starting with a software interface provided by processing circuitry 98 of server 94 via one of computing devices 100, sending the EGM in question to the computing device 100, and storing the result in storage device 96. This system will focus on ease of use and may include AI features for nurses to send the EGM to the cardiologist. This includes: (1) additional software menu provided by processing circuitry 98 of server 94 that allows the nurse to choose which EGM episodes to send to one or more cardiologists of a list of cardiologists, as well as a which questions to ask, either from a pre-defined list or open-ended; (2) a zoomed in region of interest of the EGM, based on either nurse input or AI decision, is sent to a phone-based/tablet-based application available to the cardiologist via another computing device 100, who gets an alert; and cardiologist logs into the application and is given a list view of all the indicated episodes, where they can one-click tap on to see the zoomed region of interest and answer the questions. These results are sent back to server 94 and stored automatically for the nurse to see and read. An alert notification is also sent to server 94. In some cases, the zoomed regions of interest may be relatively short selection of data, e.g., data corresponding to 10 seconds of readings, selected from a longer data reading, e.g., data corresponding to 15 minutes of readings. These zoomed regions of interest may be determined by the nurse or AI decision based on analysis of the episode data, e.g., the entire episode data. This identification of regions of interest is advantageous in that the cardiologist is able to view all the pertinent data on a phone or tablet screen without needed to scroll through excess data. Resultingly, the cardiologist may be able to analyze the data more efficiently and effectively.

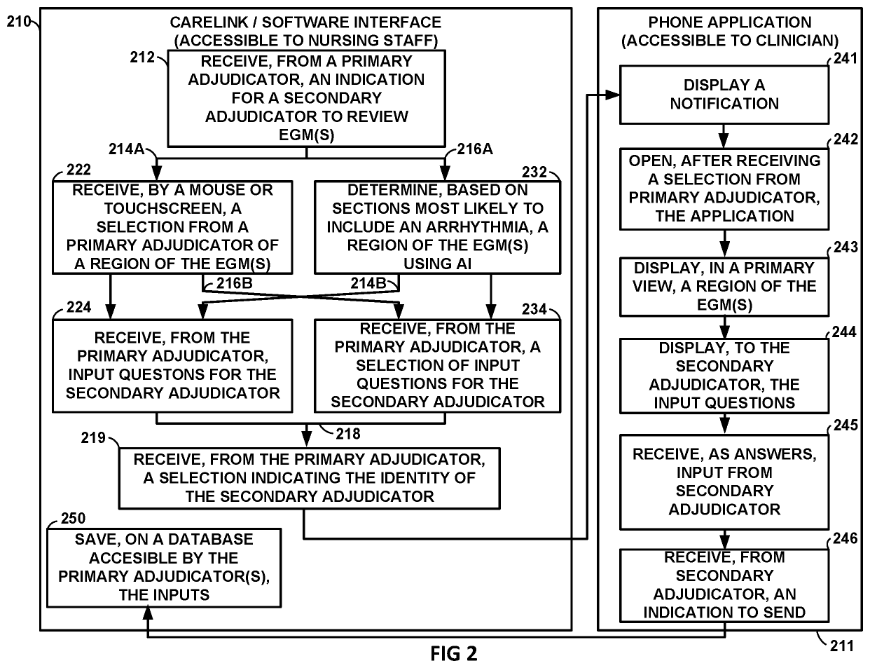

FIG. 2 is a flow diagram illustrating an example of the techniques for cardiac episode adjudication including operation of a software interface, which is accessible to primary adjudicators, and a phone application, which is accessible to clinicians, as well as some examples of information the software interface and phone application may require as inputs from both adjudicators. FIG. 2 illustrates processes that may be performed by two computing devices (e.g., computing devices 100 of FIG. 1) associated with two adjudicators, such as a nurse or other primary adjudicator, and a clinician or other secondary adjudicator. Each block illustrated in FIG. 2 may be associated with a different interface screen presented by the respective computing device. Input from the user such as clicks or the like may move to the screen, and the opportunity for additional input, e.g., text fields, may be provided. In some examples, server 94 may provide a software interface to visualize waveforms of patients, as well as save clinician's notes about patients. The aforementioned waveforms and clinician's notes are accessible by a primary adjudicator via one of computing devices 100, and another computing device associated with the secondary adjudicator may execute a mobile based application (for both phone and tablet) that is downloadable on clinicians' phones and can send notifications.

Techniques performed by CareLink™/software interface 210 may be executed by a first computing device of computing devices 100, and techniques performed by phone application 220 may be executed by a second computing device of computing devices 100. Techniques performed by CareLink™/software interface 210 may include executing instructions by processing circuity of first computing device and/or processing circuitry 98 of server 94. Techniques performed by phone application 220 may include executing instructions by processing circuitry of second computing device and/or processing circuitry 98 of server 94.

CareLink™/software interface 210 may represent techniques involving a first adjudicator (e.g., nursing staff, doctors, care givers, technicians, or specialists). In some examples, the first adjudicator may communicate with CareLink™/software interface by interacting with the first computing device of computing devices 100. Tactile and visual sensors (e.g., cameras, tactile screens, buttons, and accelerometers) may receive the actions of the first adjudicator as inputs or input data from the sensors. The input data may be sent to CareLink™/software interface 210 for further analysis.

CareLink™/software interface 210 may receive, from the primary adjudicator, an indication for a secondary adjudicator to review EGM(s) (212). The indication that EGM(s) should be reviewed, may include the primary adjudicator selecting an image, text, or icon indicating his/her intention to have one or more EGM(s) submitted to a second adjudicator for review. In some examples, to indicate one or more EGM(s) should be reviewed, CareLink™/software interface 210 may allow the user (e.g., primary adjudicator) to capture an image or upload a data file representing an EGM measurement. CareLink™/software interface 210 may allow the user to select a region of the EGM(s) with manual 214A techniques or automatic 216A techniques, to indicate the EGM is ready for review.

In some examples, manual 214A techniques may be used to select EGM(s). For example, CareLink™/software interface may receive, by a mouse or touchscreen, a selection from a primary adjudicator of a region of the EGM(s) (222). The primary adjudicator may select a region of the EGM(s) using the mouse or touchscreen a region of an EGM displayed on one of computing devices 100A-100N. Selecting may include touching a touch screen and dragging across the region of the EGM and releasing at an edge of the region.

The primary adjudicator may, in some examples, choose to manually type a question regarding the event data within the input form of the CareLink™/software interface 210. In some examples, the primary adjudicator may select from a list of AI-generated questions based on the event episode data and specific arrhythmia detected.

In some examples, additionally or alternatively, automatic 216A techniques may be used to determine regions of EGM(s) for review. For example, CareLink™/software Interface may be configured to determine, based on sections most likely to include an arrhythmia, a region of the EGM(s) using AI, e.g., an ML model (232). AI may compare and weight a plurality of inputs based on a plurality of regions of the EGM(s) to generate a cost function. AI may minimize the cost function to arrive at the region of the EGM to select. AI may be configured to run on server 94 and/or computing devices 100A-100N.

In some examples, manual techniques may be switched to automatic techniques 216B and automatic techniques may be switched to manual techniques 214B. For example, after the primary adjudicator selects a region of the EGM, the primary adjudicator may request for the CareLink™/software interface to assist in generating questions for the clinician. Alternatively, after the AI selects sections of the EGM, the primary adjudicator may write questions within an input form of the CareLink™/software interface manually. In some examples, the primary adjudicator may choose to manually select a region of the EGM and may also manually write questions within the input form of the CareLink™/software interface. Alternatively, the primary adjudicator may decide to have the AI select a region of the EGM and may also decide to write questions within the input form of the interface manually.

When the interface is configured for manual techniques, CareLink™/software interface may be configured to receive, from the primary adjudicator, input questions for the secondary adjudicator (224). In some examples, the input questions may include questions manually entered in an input box of an input form. Input questions may include text or written characters for review by the secondary adjudicator.

When the interface is configured for automatic techniques, CareLink™/software interface may be configured to receive, from the primary adjudicator, a selection of input questions for the secondary adjudicator (234). The selection may include selecting proposed questions generated by CareLink™/software interface using AI. In some examples, selecting may include the following: highlighting text, selecting a checkbox, selecting a link, or otherwise providing input indicating the primary adjudicator's intent to select a question.

Whether the primary adjudicator has used manual techniques or automatic techniques, CareLink™/software interface may be configured to receive, from the primary adjudicator, a selection indicating the identity of the secondary adjudicator (219). For example, the primary adjudicator may select the identity of the second adjudicator by entering an email, selecting a name, selecting a contact, or providing another type of input indicating the primary adjudicator's intent to identify a secondary adjudicator.

Identifying a secondary adjudicator may cause a phone application 211 to execute instructions on one or more computing device(s) 100A-100N. Phone application 211 may be configured to display a notification (241). In some examples, the notification may be displayed on one or more of computer devices 100A-100N, based on the primary adjudicator's identification of the secondary adjudicator.

Phone application 211 may be further configured to display, in a primary view, a region of the EGM(s) (243). Displaying may include presenting an image of the region of the EGM on the screen, display, or touchscreen, or a computing device. The computing device may include a computing device of computing devices 100A-100N.

Phone application 211 may be configured to display, to the secondary adjudicator, the input questions (244). In some examples, displaying the input questions may include displaying input questions received from primary adjudicator. For example, received questions or selection of questions from the primary adjudicator may correspond to questions displayed to the secondary adjudicator.

Phone application 211 may be configured to receive, as answers, inputs from secondary adjudicator (245). In some examples, answers may include observations, comments, and direct responses to the input questions. Phone application 211 may receive the inputs from one of computing devices 100A-100N via touchscreen, keyboard, voice to text, or another user interface.

In various examples, phone application 211 may be configured to receive, from secondary adjudicator, an indication to send (246). The indication to send may include an input indicating the secondary adjudicator's intent to send the inputs back to the primary adjudicator. In some examples, the indication may be received by selecting a button with the word “send” written on it. In some examples, the indication may include receiving a voice command, selecting an icon, swiping on a touchscreen, or receiving a different input from the user indicating the user's intent to send the inputs.

In response to receiving a send indication, CareLink™/software interface may be configured to save the inputs (250) on a database accessible by primary adjudicators. The inputs may include inputs as answers received from the secondary adjudicator, addressing questions provided or selected by the primary adjudicator. The database may include server 94 saving the inputs on storage device 96.

In various examples, processing or instruction execution on CareLink™/software interface and on phone application 211 may be completed in whole or in part on server 94, network 92, and computing devices 100A-100N.

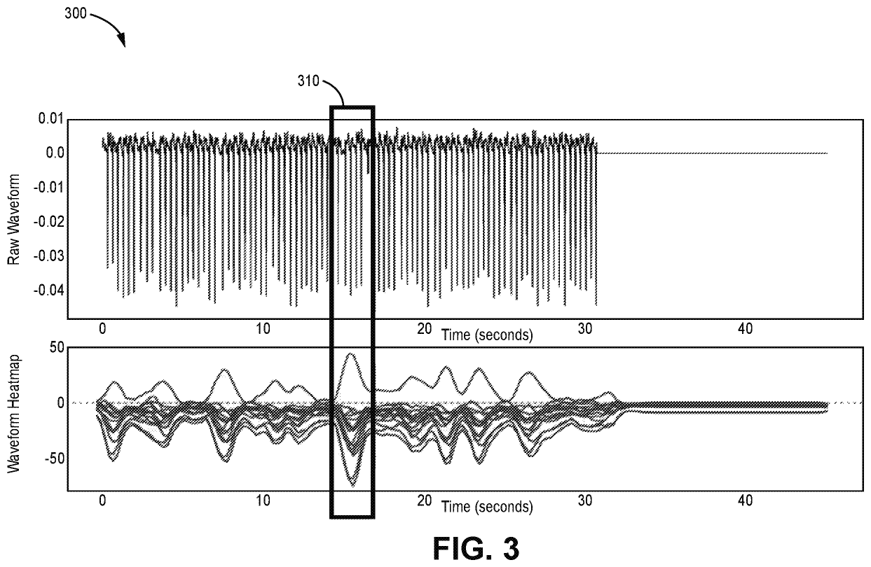

FIG. 3 is a diagram illustrating an example of cardiac EGM data 300 indicating a selection of a region including an episode, in accordance with one or more techniques of this disclosure. The EGM data 300 may include a plurality of data points taken over time indicating a state of the heart at the time the signal was sensed by IMD 10. The graphical representation of EGM data 300 over a time period may illustrate the presence of an episode. In some examples, the presence of an episode may be illustrated by an abnormality in the EGM data 300. While a primary adjudicator may not be able to definitively diagnose an episode, the presence of an abnormality in the illustration of EGM data 300 may provide an indication on a region 310 over which to select for further review.

In some examples, the selection of region 310 may be made automatically through artificial intelligence (AI). Processing circuitry 98 of server 94 may apply a machine learning model or other artificial intelligence analysis to the EGM and in some cases other episode data to determine the likelihoods of different arrhythmia classifications over time. In some examples, processing circuitry 98 may implement the techniques of commonly-assigned U.S. patent application Ser. No. 16/845,996, filed Apr. 10, 2020, titled “MACHINE LEARNING BASED DEPOLARIZATION IDENTIFICATION AND ARRHYTHMIA LOCALIZATION VISUALIZATION,” the entire content of which is incorporated herein by reference. Processing circuitry 98 may determine a region of the episode data associated with a suprathreshold likelihood of an arrhythmia classification, e.g., atrial fibrillation (AF) in region 310 of FIG. 3. Processing circuitry 98 may cause one or more interfaces herein to present region 310 zoomed in for review by an adjudicator. In some examples, a user, e.g., a primary adjudicator, may additionally or alternatively determine a region of interest which may be presented to another user, e.g., a secondary adjudicator. In this case, the primary adjudicator may select a region 310 of EGM data 300 by clicking and dragging across the region of interest or otherwise indicating the user's intent to send the inputs.

Example machine learning techniques that may be employed to select region 310 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

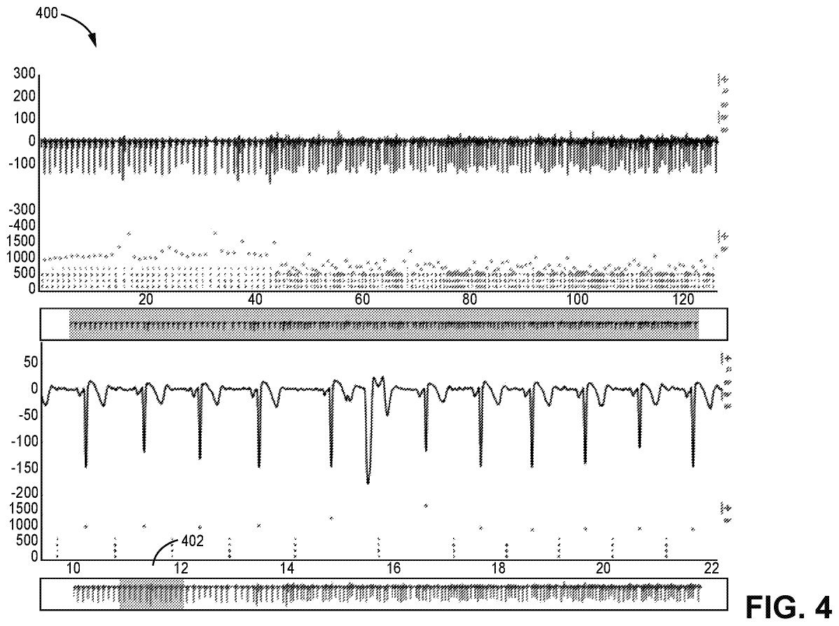

FIG. 4 is a conceptual diagram illustrating an example user interface including a zoomed in region 400 of the episode data, e.g., as discussed with respect to FIG. 3. For example, zoomed in region 400 may include data points illustrated in region 310 of FIG. 3. FIG. 4 illustrates a portion of EGM data 300 plotted over time, where the portion of EGM 300 data not included in region 310 is omitted to streamline and increase efficiency of adjudication. FIG. 4 also illustrates a rate plot, e.g., a plot of R-R intervals over the time period of the episode, which may increase the efficiency and speed of adjudications.

In some examples, the user (e.g., primary adjudicator) may be able to scroll through EGM data 310 by moving the highlighted portion 402, e.g., using the bottom portion of the interface of FIG. 4 as a scroll bar. The primary adjudicator, may select or highlight the portion or region of EGM data 300 by selecting, dragging, and releasing his/her hand over a touch screen of a device of computing devices 100A-100N. In some examples, the user (e.g., primary adjudicator) may pan or scroll to another section of EGM data 300 containing the episode. The selection may be zoomed in on the display of the computing device used by the user. The region initially displayed as zoomed may be selected by processing circuitry, e.g., processing circuitry 98 and/or by application of a machine learned model as described above, or by another user.

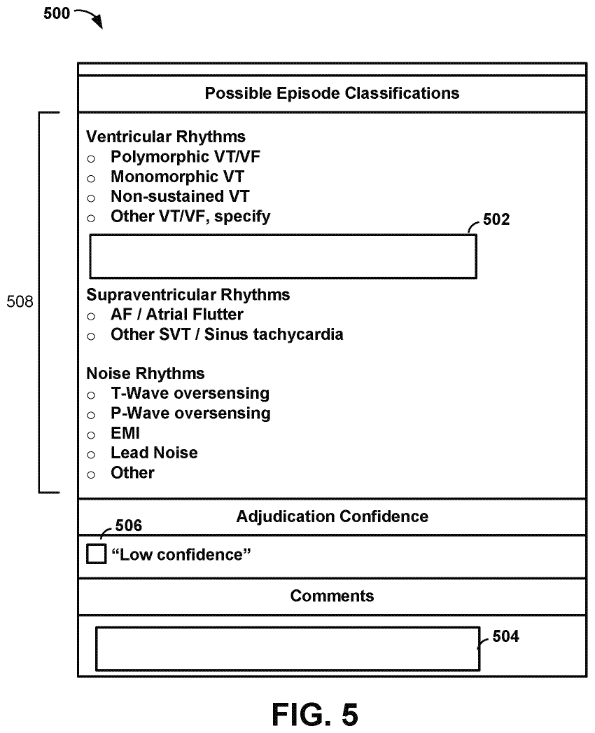

FIG. 5 is a conceptual diagram illustrating an example user interface including an input form for receiving inputs from a primary adjudicator, in accordance with one or more techniques of this disclosure. Based on review of the episode, e.g., via the interface shown in FIG. 4, a user, e.g., a nurse of other primary adjudicator, may be presented with the question list 508 shown in FIG. 5 to provide input on the adjudication. In the event the classification is not contained in the list, input field 502 may allow the primary adjudicator to input another classification.

FIG. 5 also contains an adjudication confidence selection (506), where the primary adjudicator may indicate, via an input, a confidence level associated with the initial adjudication. Additionally, comment input box 504 may allow the primary adjudicator to send specific comments to the secondary adjudicator regarding the episode data.

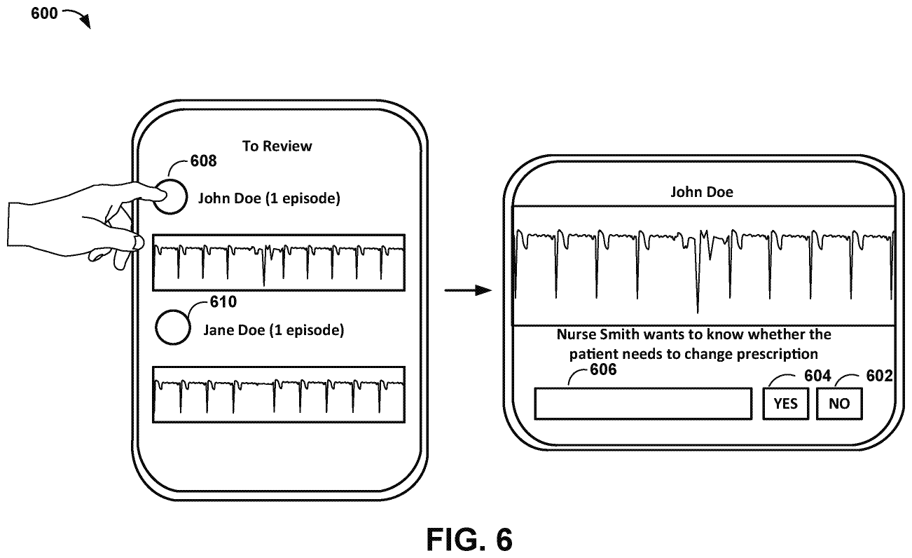

FIG. 6 illustrates example interfaces for a secondary adjudicator. As illustrated in FIG. 6, the secondary adjudicator, e.g., cardiologist or other clinician, may be presented with a list of or notifications for patient(s) and episode(s) of the patient(s) for further review. The secondary adjudicator, may select patient(s) and episode(s) of the patient(s) by pressing a touch screen of a computing device 100, selecting display button 608 and/or display button 610. Upon the selection of display button(s), the computing device 100 may then display on the screen an interface including a section of the episode data selected by the primary adjudicator/AI and prompting the secondary adjudicator to select a response to the primary adjudicator's input query. In some examples, data processing for selection of an episode region will come in the form of backend AI algorithm or machine learning model deployment to run on the EGMs and return the region in question, followed by additional digital processing and visualization, e.g., on the computing device 100, to show that region. The output is the view for the nurse and clinician and a relatively quick turnaround for the adjudications via system 2. The secondary adjudicator may type a response in comment box 606 and/or may select “yes” button 604 or “no” button 602.

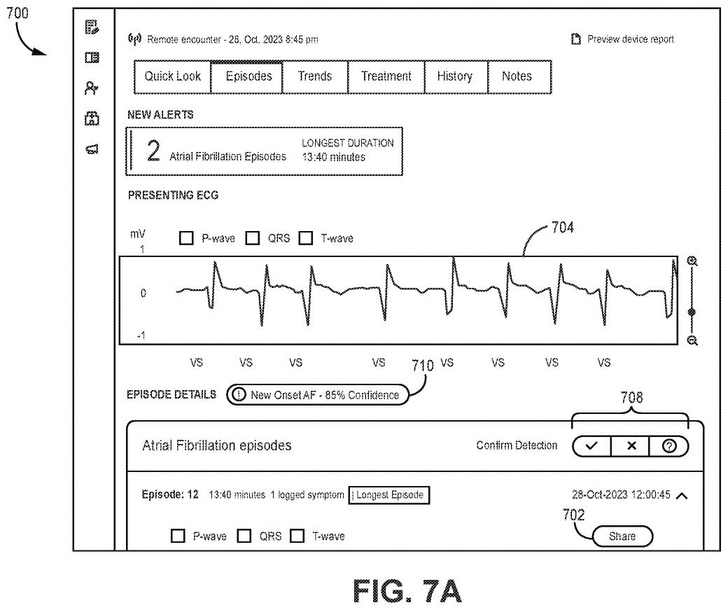

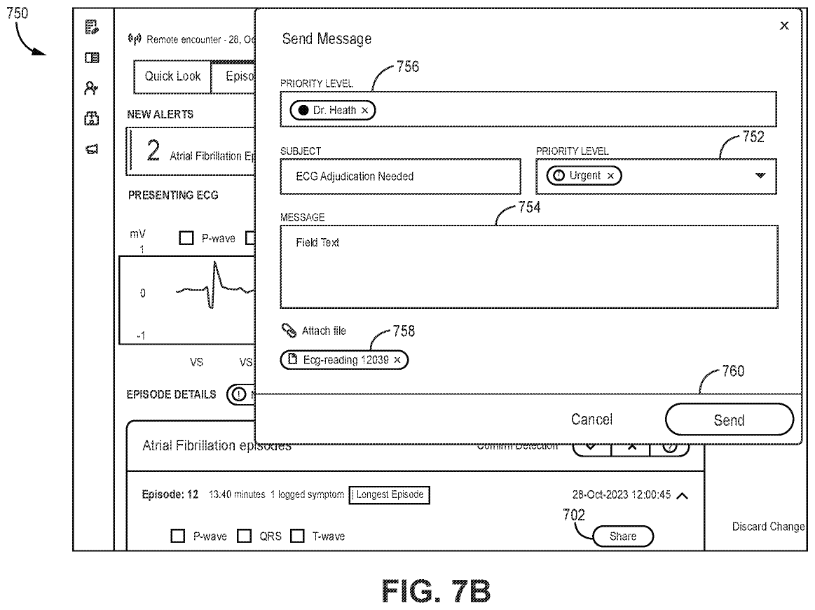

FIG. 7A is a conceptual diagram illustrating an example interface 700 for primary adjudicators to make initial adjudications based on episode data and to decide whether to send episode data to a secondary adjudicator. As illustrated in FIG. 7A, an episode, e.g., an atrial fibrillation episode, may be flagged for investigation. The interface may indicate the confidence level associated with the episode(s) that have occurred via confidence notification 710. The interface may additionally include a graphical representation 704 of episode data. The primary adjudicator may use this information to determine whether the patient experienced the cardiac arrhythmia associated with the flagged episodes and select from detection confirmation options 708, or the primary adjudicator may send this information to the secondary adjudicator for further review by selecting share button 702.

FIG. 7B is a conceptual diagram illustrating an example pop-up interface 750 resulting from the selection of share button 702 in the interface of FIG. 7A. In some examples, the primary adjudicator may be able to select a priority level 752 to indicate the urgency with which the secondary adjudicator is advised to examine the episode data. In recipient field 756, the primary adjudicator may enter the name of the desired recipient. The episode data file may be attached to the message using attach file button 758. The episode data file may include zoomed in regions of interest. These regions may be selected using AI, primary adjudicator input, or a combination thereof. In some examples, the primary adjudicator may be prompted to send an individualized message in message field 754 along with the data. The primary adjudicator may send this message using send button 760.

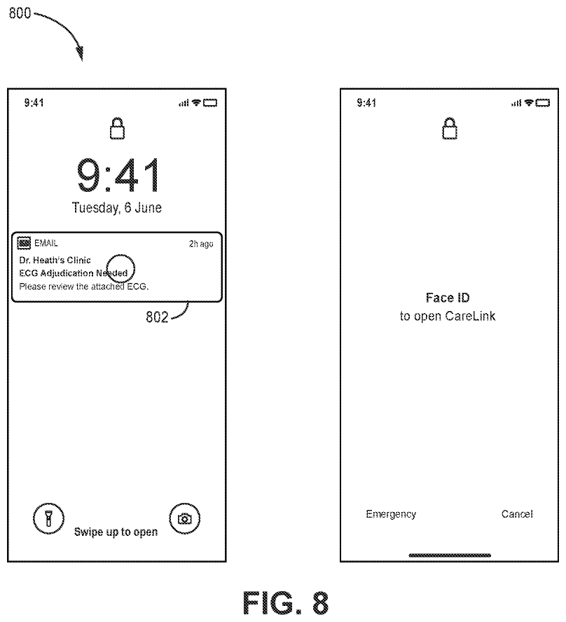

FIG. 8 is a conceptual diagram illustrating the interface of and process by which a secondary adjudicator receives a notification that a new EGM adjudication is needed and securely accesses a computing device 100 to review the EGM. In some examples, the secondary adjudicator may be notified via email. The secondary adjudicator may initiate sign in on a touch screen device by selecting notification 802. In some examples, the secondary adjudicator may access the data using his/her phone login credentials, e.g., Face ID. Upon unlocking the device, the secondary adjudicator gains access to the data available in the web browser. In some examples, the secondary adjudicator may use the phone/tablet application rather than the web browser, depending on user preference. The secondary adjudicator may then examine the data and assess the data.

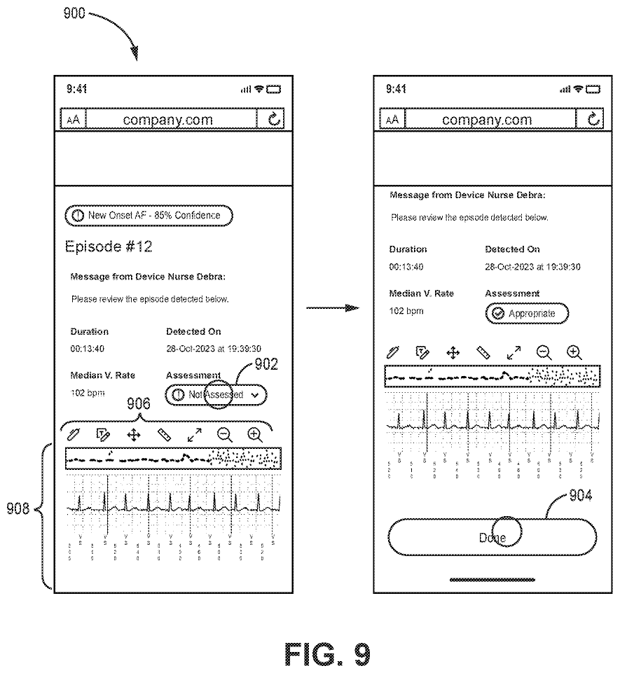

FIG. 9 is a block diagram illustrating an example interface 900, in which episode data is accessed via a secure web browser. In some examples, the secondary adjudicator may use virtual examination tools 906 to aid in assessment, e.g., the secondary adjudicator may zoom in, zoom out, measure, highlight, etc., of episode data 908, e.g., the segment of episode data identified by the primary adjudicator or AI model for review. In some examples, the secondary adjudicator may select an assessment from a dropdown list 902. At the conclusion of the examination, the secondary adjudicator may select “DONE” button 904, which may initiate the transfer of the secondary adjudicator's assessment to database 250 of system 2.

In some examples, the techniques of the disclosure include a system that comprises means to perform any method described herein. In some examples, the techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

This disclosure includes the following non-limiting examples.

### EXAMPLE 1

A computing system comprising memory storing instructions and processing circuitry of one or more devices, including a server, a computing device of a primary adjudicator, and a computing device of a secondary adjudicator, configured to execute those instructions to: receive, at the server, episode data of a cardiac episode from a medical device; identify, by the server, a region of the episode data; provide, via the computing device of the secondary adjudicator, a notification to the secondary adjudicator; display, in response to the secondary adjudicator interacting with the computing device of the secondary adjudicator in response to the notification, the region of the episode data onto the display of the computing device of the secondary adjudicator; receive, by the computing device of the secondary adjudicator, an input; and transmit, by the computing device of the secondary adjudicator and to the server, the input received from the secondary adjudicator in response to the episode data.

### EXAMPLE 2

The computing system of example 1, wherein the episode data comprises cardiac electrogram or electrocardiogram data.

### EXAMPLE 3

The computing system of any one or more of examples 1 or 2, wherein the processing circuitry is configured to apply the episode data to a machine learning model to determine the region

### EXAMPLE 4

The computing system of any one or more of examples 1 to 3, wherein the server is configured to determine based on an input from a primary adjudicator, the region of the episode data.

### EXAMPLE 5

The computing system of any one or more of examples 1 to 4, wherein the computing device of the secondary adjudicator is configured to display the notification on a screen of the computing device of the secondary adjudicator.

### EXAMPLE 6

The computing system of any one or more of examples 1 to 5, wherein the server is configured to save the input received from the secondary adjudicator.

### EXAMPLE 7

The computing system of any one or more of examples 1 to 6, wherein the computing device of the primary adjudicator is configured to receive an input question from the primary adjudicator, wherein the server is configured to cause the computing device of the secondary adjudicator to present the input question to the secondary adjudicator.

### EXAMPLE 8

The computing system of example 7, wherein the computing device of the primary adjudicator is configured to receive, from the primary adjudicator, a selection from among input questions determined by application of the episode data to a machine learning model.

### EXAMPLE 9

The computing system of any one or more of examples 1 to 7, wherein the computing device of the primary adjudicator is configured to receive, from the primary adjudicator, a selection indicating the identity of the secondary adjudicator.

### EXAMPLE 10

The computing system of any one or more of examples 1 to 9, wherein to display the region the computing device of the secondary adjudicator is configured to zoom in on the region.

### EXAMPLE 11

A method of operating a computing system to facilitate adjudication of cardiac events between a primary adjudicator and a secondary adjudicator, the method comprising: receiving, at a server, episode data of a cardiac episode from a medical device; identifying, by the server, a region of the episode data; providing, via a computing device of the secondary adjudicator, a notification to the secondary adjudicator; displaying, in response to the secondary adjudicator interacting with the computing device of the secondary adjudicator in response to the notification, the region of the episode data onto the display of the computing device of the secondary adjudicator; receiving, by the computing device of the secondary adjudicator, an input; and transmitting, by the computing device of the secondary adjudicator and to the server, the input received from the secondary adjudicator in response to the episode data.

### EXAMPLE 12

The method of example 11, wherein the episode data comprises cardiac electrogram or electrocardiogram data.

### EXAMPLE 13

The method of any one or more of examples 11 or 12, further comprising, by the processing circuitry, applying the episode data to a machine learning model to determine the region.

### EXAMPLE 14

The method of any one or more of examples 11 to 13, further comprising determining, by the server and based on an input from a primary adjudicator, the region of the episode data.

### EXAMPLE 15

The method of any one or more of examples 11 to 14, further comprising displaying the notification on a screen of the computing device of the secondary adjudicator.

### EXAMPLE 16

The method of any one or more of examples 11 to 15, further comprising saving the input received from the secondary adjudicator by the server.

### EXAMPLE 17

The method of any one or more of examples 11 to 16, further comprising receiving, by the computing device of the primary adjudicator, an input question from the primary adjudicator, wherein the server is configured to cause the computing device of the secondary adjudicator to present the input question to the secondary adjudicator.

### EXAMPLE 18

The method of example 17, further comprising receiving, by the computing device of the primary adjudicator from the primary adjudicator, a selection from among input questions determined by application of the episode data to a machine learning model.

### EXAMPLE 19

The method of any one or more of examples 11 to 17, further comprising receiving, by the computing device of the primary adjudicator from the primary adjudicator, a selection indicating the identity of the secondary adjudicator.

### EXAMPLE 20

The method of any one or more of examples 11 to 19, wherein displaying the region comprises zooming in on the region.

### EXAMPLE 21

A non-transitory computer readable storage medium comprising program instructions that, when executed by processing circuitry of a computing system comprising a server, a computing device of a primary adjudicator, and a computing device of a secondary adjudicator, cause the processing circuitry to perform a method comprising: receiving, at the server, episode data of a cardiac episode from a medical device; identifying, by the server, a region of the episode data; providing, via a computing device of the secondary adjudicator, a notification to the secondary adjudicator; displaying, in response to the secondary adjudicator interacting with the computing device of the secondary adjudicator in response to the notification, the region of the episode data onto a display of the computing device of the secondary adjudicator; receiving, by the computing device of the secondary adjudicator, an input; and transmitting, by the computing device of the secondary adjudicator and to the server, the input received from the secondary adjudicator in response to the episode data.

Various examples have been described. These and other examples are within the scope of the following claims.

1. A computing system comprising memory storing instructions and processing circuitry of one or more devices, including a server, a computing device of a primary adjudicator, and a computing device of a secondary adjudicator, configured to execute those instructions to:
receive, at the server, episode data of a cardiac episode from a medical device;
identify, by the server, a region of the episode data;
provide, via the computing device of the secondary adjudicator, a notification to the secondary adjudicator;
display, in response to the secondary adjudicator interacting with the computing device of the secondary adjudicator in response to the notification, the region of the episode data onto a display of the computing device of the secondary adjudicator;
receive, by the computing device of the secondary adjudicator, an input; and
transmit, by the computing device of the secondary adjudicator and to the server, the input received from the secondary adjudicator in response to the episode data.

2. The computing system of claim 1, wherein the episode data comprises cardiac electrogram or electrocardiogram data.

3. The computing system of claim 1, wherein the processing circuitry is configured to apply the episode data to a machine learning model to determine the region.

4. The computing system of claim 1, wherein the server is configured to determine based on an input from the primary adjudicator, the region of the episode data.

5. The computing system of claim 1, wherein the computing device of the secondary adjudicator is configured to display the notification on a screen of the computing device of the secondary adjudicator.

6. The computing system of claim 1, wherein the server is configured to save the input received from the secondary adjudicator.

7. The computing system of claim 1, wherein the computing device of the primary adjudicator is configured to receive an input question from the primary adjudicator, wherein the server is configured to cause the computing device of the secondary adjudicator to present the input question to the secondary adjudicator.

8. The computing system of claim 7, wherein the computing device of the primary adjudicator is configured to receive, from the primary adjudicator, a selection from among input questions determined by application of the episode data to a machine learning model.

9. The computing system of claim 1, wherein the computing device of the primary adjudicator is configured to receive, from the primary adjudicator, a selection indicating the identity of the secondary adjudicator.

10. The computing system of claim 1, wherein to display the region the computing device of the secondary adjudicator is configured to zoom in on the region.

11. A non-transitory computer readable storage medium comprising program instructions that, when executed by processing circuitry of a computing system comprising a server, a computing device of a primary adjudicator, and a computing device of a secondary adjudicator, cause the processing circuitry to perform a method comprising:
receiving, at the server, episode data of a cardiac episode from a medical device;
identifying, by the server, a region of the episode data;
providing, via a computing device of the secondary adjudicator, a notification to the secondary adjudicator;
displaying, in response to the secondary adjudicator interacting with the computing device of the secondary adjudicator in response to the notification, the region of the episode data onto a display of the computing device of the secondary adjudicator;
receiving, by the computing device of the secondary adjudicator, an input; and
transmitting, by the computing device of the secondary adjudicator and to the server, the input received from the secondary adjudicator in response to the episode data.

12. A method comprising:
receiving, at a server of a computing system comprising memory storing instructions and processing circuitry of one of more devices, including the server, a computing device of a primary adjudicator and a computing device of a secondary adjudicator, episode data of a cardiac episode from a medical device;
identifying, by the server, a region of the episode data;
providing, via the computing device of the secondary adjudicator, a notification to the secondary adjudicator;
displaying, in response to the secondary adjudicator interacting with the computing device of the secondary adjudicator in response to the notification, the region of the episode data onto a display of the computing device of the secondary adjudicator;
receiving, by the computing device of the secondary adjudicator, an input; and
transmitting, by the computing device of the secondary adjudicator and to the server, the input received from the secondary adjudicator in response to the episode data.

13. The method of claim 12, wherein the episode data comprises cardiac electrogram or electrocardiogram data.

14. The method of claim 12, wherein the processing circuitry is configured to apply the episode data to a machine learning model to determine the region.

15. The method of claim 12, wherein the server is configured to determine based on an input from the primary adjudicator, the region of the episode data.

16. The method of claim 12, wherein the computing device of the secondary adjudicator is configured to display the notification on a screen of the computing device of the secondary adjudicator.

17. The method of claim 12, wherein the server is configured to save the input received from the secondary adjudicator.

18. The method of claim 12, further comprising:
receiving, by the computing device of the primary adjudicator, an input question from the primary adjudicator; and
causing, by the server, the computing device of the secondary adjudicator to present the input question to the secondary adjudicator.

19. The method of claim 18, further comprising:
receiving, by the computing device of the primary adjudicator and from the primary adjudicator, a selection from among input questions determined by application of the episode data to a machine learning model.

20. The method of claim 12, further comprising:
receiving, the computing device of the primary adjudicator and from the primary adjudicator, a selection indicating the identity of the secondary adjudicator.
